(19) United States
(12) Patent Application Publication
(10) Pub. No.: US 2021/0325357 A1
(43) Pub. Date: Oct. 21, 2021

(54) Flow Controller and Method of Use

(71) Applicant: Entech Instruments Inc., Simi Valley, CA (US)

(72) Inventor: Daniel B. CARDIN, Simi Valley, CA (US)

(21) Appl. No.: 17/231,365

(22) Filed: Apr. 15, 2021

Related U.S. Application Data
(60) Provisional application No. 63/011,574, filed on Apr. 17, 2020.

(51) Int. Cl. G01N 33/00; G05D 7/01

(52) U.S. Cl. CPC G01N 33/0006; G05D 7/012

(57) ABSTRACT

A flow controller for filling evacuated canisters can be operated at different reference pressures to produce substantially the same flow rates to facilitate inertness testing of the flow controller through demonstrated recovery of trace level chemicals in a challenge standard prior to using the flow controllers to collect air samples for measurement of VOCs during time weighted sampling events. The flow controller can include a first chamber and a second chamber divided by a diaphragm. The first chamber can be fluidly coupled to an inlet of the flow controller and an outlet of the flow controller. The second chamber can be coupled to a reference port of the flow controller. The outlet of the flow controller can be coupled to an initially (e.g., substantially) evacuated canister that can be used to collect a sample of ambient air or challenge standard (e.g., during testing).

### CROSS-REFERENCE TO RELATED APPLICATION

This application claims the benefit of U.S. Provisional Patent Application No. 63/011,574, filed on Apr. 17, 2020, the entire disclosure of which is incorporated herein by reference in its entirety for all purposes.

### FIELD OF THE DISCLOSURE

This relates to a flow controller and, more particularly, to a flow controller for controlling the flow of gas into an initially evacuated canister.

### BACKGROUND OF THE DISCLOSURE

Collection of whole air samples into pre-evacuated canisters is a popular way to collect air samples for the measurement of VOCs. An initial high vacuum inside of stainless steel canisters ranging in volume from 1-15 L (e.g., 6 L) provides a sample collection device that is initially free of any VOC background, while also providing the driving force to “draw” air into the canister during sampling of atmospheric pressure air. When these canisters are taken into the field, the isolation valve can be opened up to rapidly fill the canister in 0.1-1 min, which is known as a “grab sample”. However, considering that concentrations of VOCs in air depend on multiple parameters, including the location and distance of VOC emission sources, and the current meteorological conditions (wind speed, wind direction, rain, rising pressure, falling pressure, etc.), collecting a grab sample will usually not provide a good representation of the typical or average VOC concentration in any given location. The average concentration of VOCs at a respective location is important, as an average provides a better assessment of risk for people living or working in these areas than the assessment provided by a grab sample. Generally, at ambient levels, VOCs do not present an acute risk of suffocation, but do present a threat for long term, chronic illness such as heart disease and cancer due to the inflammatory and carcinogenic nature of many VOCs. The US EPA has created risk levels associated with more than 100 VOCs, which indicate the relative chances of getting cancer when exposed to different VOCs over a long period of time. These risk levels vary tremendously and can be very low for some compounds (Methane and Propane, for example), yet very high for other compounds (1,3-Butadiene, Benzene, Vinyl Chloride, and many others). It is therefore important to be able to determine the concentration of individual chemicals over an extended period of time rather than simply relying on VOC snapshots provided by grab samples. Drawing an air sample into a canister very slowly and at a constant rate creates a time weighted average of the air at a respective location, allowing a composite concentration to be obtained in the canister for measurement later in a mobile or stationary laboratory. This average concentration provides a far better assessment of risk, as the average exposure concentrations will affect the average amount that is building up in the human body, considering these compounds are both absorbed and outgassed from the body over time.

### SUMMARY OF THE DISCLOSURE

This relates to a flow controller and, more particularly, to a flow controller for controlling the flow of gas into an initially evacuated canister. In some embodiments, the flow controller can be used for filling evacuated canisters to measure VOCs in indoor and outdoor air at PPM, PPB, and sub-PPB levels. The disclosed flow controller enables a quality assurance testing setup that enables verification of the recovery of VOCs during sampling. The flow controller uses a pressure balancing technique to create substantially the same flow rate from a pressurized challenge gas mixture as that obtained when sampling air from a non-pressurized source such as ambient air. Embodiments of the disclosure can maintain consistent flow rates and therefore resonance times, thereby enabling the validation of sample recovery, as adsorption/absorption and reactions can vary based on exposure times within the sampler.

### BRIEF DESCRIPTION OF THE DRAWINGS

FIG. 1 illustrates a flow controller according to some embodiments.

FIGS. 2A-2B illustrate a test setup including a flow controller according to some embodiments of the disclosure.

FIGS. 3A-3B illustrate a sampling system including a flow controller according to some embodiments of the disclosure.

FIG. 4 illustrates a method of challenging a flow controller for proper recovery of target compounds of interest according to some embodiments of the disclosure.

FIG. 5 illustrates a method of collecting a sample using flow controller according to some embodiments of the disclosure.

### DETAILED DESCRIPTION

In the following description, reference is made to the accompanying drawings which form a part hereof, and in which it is shown by way of illustration specific examples that can be practiced. It is to be understood that other examples can be used and structural changes can be made without departing from the scope of the examples of the disclosure.

This relates to a flow controller and, more particularly, to a flow controller for controlling the flow of gas into an initially evacuated canister. In some embodiments, the flow controller can be used for filling evacuated canisters to measure VOCs in indoor and outdoor air at PPM, PPB, and sub-PPB levels. The disclosed flow controller enables a quality assurance testing setup that enables verification of the recovery of VOCs during sampling. The flow controller uses a pressure balancing technique to create substantially the same flow rate from a pressurized challenge gas mixture as that obtained when sampling air from a non-pressurized source such as ambient air. Embodiments of the disclosure can maintain consistent flow rates and therefore resonance times, thereby enabling the validation of sample recovery, as adsorption/absorption and reactions can vary based on exposure times within the sampler.

### Introduction

Mechanical flow controllers can be used to perform time weighted average sampling. In some embodiments, mechanical flow controllers operate as “sub-atmospheric back pressure regulators”, referencing atmospheric pressure both at the entrance to the flow controller, and on the backside of a flow-controlling diaphragm. The flow path and materials of construction inside of these flow controllers can be important to allow VOCs to travel through them without adsorbing or absorbing onto or into surfaces of the flow controller. In addition, surfaces inside of the flow controllers should be (e.g., fully, substantially, partially etc.) inert to avoid the reaction of target VOCs with these surfaces, so that recovery of sample through the flow controller is as close to 100% as possible. In some embodiments, over time, flow controllers may become contaminated with heavier compounds (e.g., semi-volatile organic compounds) or particulates that can also reduce the recovery of compounds during sampling, resulting in the absorption of target VOCs, and the ultimate reduction in recoveries into the receiving canister.

In the past, various agency methods have not required challenging these flow controllers for recovery at the same flow rates to be used during field sampling. However, US EPA Method TO-15A released in early 2020 now requires the verification of VOC target compound recovery through the sample collection flow path (sample train) to verify recovery of VOCs, both before implementation of the sample trains to collect real air samples, and thereafter at regular intervals (every 3 years, after disassembly for cleaning, or after exposure to a high concentration sample). This requirement creates a challenge for current sample trains, as these sample trains generally require atmospheric pressure at the inlet of the flow controller in order to achieve a flow rate similar to the flow rate during sampling. Connecting a pressurized standard to a flow controller to test the flow controller will typically result in a flow rate that is 300% to 2000% higher than the flow rate achieved at atmospheric pressure. This change in flow rate changes the residence time of the VOCs in the flow controller and/or sample train, for example. Thus, challenging a flow controller with a pressurized challenge standard may not accurately reflect VOC recovery under actual field sampling events where VOC exposure to the inside of the flow controller could be much longer due to the lower flow rates through the flow controller.

In the new TO-15A method, the EPA requires that flow controllers be tested at or near the same flow rates as they will be used at in the field. Therefore, a direct connection to a pressurized standard cannot be used during testing unless the flow rates of the challenge standard through the flow controller can be similar to the flow rates through the flow controller when sampling air at atmospheric pressure. To test current atmospheric pressure mechanical flow controllers, a suggested approach described in US EPA Method TO-15A uses an electronic mass flow controller (MFC) connected to a challenge standard canister or cylinder to maintain a flow at atmospheric pressure past the inlet of the flow controller to be tested, at a flow rate a little faster than the sampling flow rate of the mechanical flow controller, with the excess flow going through a flow meter or a bubbler to verify the excess flow. This approach is difficult to operate, wasteful of expensive calibration standard gas, difficult to use on a plurality of sample trains simultaneously, and may require more fittings and tubing in the flow path (e.g., compared to the approach disclosed herein) that can also affect VOC recovery. Moreover, transferring the calibration standard to the flow controller through a mass flow controller may cause one or more of the compounds of the challenge standard to be retained or outgassed by the flow controller, thereby changing the concentration of one or more compounds of the challenge standard before the challenge standard reaches the flow controller to be tested.

Flow controllers according to embodiments of the disclosure can eliminate the need to use an atmospheric pressure challenge standard to achieve substantially the same flow rate and VOC residence time within the flow controller as those that occur during field sampling. Flow controllers according to embodiments of the disclosure can reference a VOC challenge standard at an elevated pressure at the inlet of the flow controller and on the backside of a control diaphragm to create nearly the same flow rate through the controller as obtained during atmospheric sampling. This configuration can simplify the validation of EPA Method TO-15A and other canister methods for VOC recovery testing through canister sample trains. This same approach can also be used to challenge the flow controllers with humidified zero air (VOC-free air) from a pressurized source to verify the lack of contamination within the sample train (e.g., the flow controller, tubing, fittings, etc.), which would otherwise create a positive bias in the results, making it appear that pollution levels are higher than they really are.

Flow controllers can be used to fill evacuated canisters at a constant rate over a period of time, thereby providing an average concentration during the sampling period. The flow controller can allow sampling flow rates as low as 0.15 cc/min, which can fill a 6 L canister over a period as long as 1 month. Collecting as few as one sample per month from a respective monitoring site can provide the same average concentration of target compounds from a respective location as a real time analyzer conducting hundreds of analyses that are averaged over a predetermined period of time. In some embodiments, the ability to time integrate the sample into a canister for later analysis in the lab can allow one lab analyzer to run samples from a plurality of locations, resulting in a monitoring program that can cost 1-3% of the cost of implementing real time, on-site analyzers at each location. When assessing long-term risks associated with VOCs present in air at a respective location, collecting and analyzing time-averaged samples can be preferable to deploying real-time analyzers, which can be temperamental when run outside of a laboratory setting. Implementation of a simple, no power flow controller/vacuum canister combination that can be analyzed and replaced once or twice a month can be a more accurate way of performing risk assessment around and within communities compared to the use of real-time analyzers. However, for VOCs often in concentrations in the range of PPB to sub-PPB in ambient air, it can be important to test the flow controller and/or sample train for both positive and negative bias, and the new US EPA Method TO-15A includes this requirement.

### Exemplary Systems and Processes

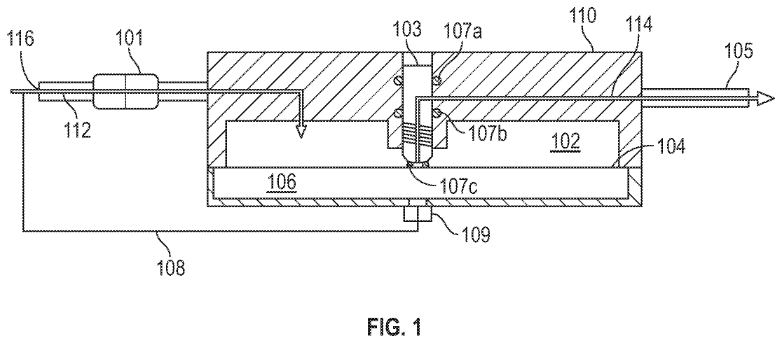

FIG. 1 illustrates a flow controller 100 according to some embodiments. In some embodiments, the flow controller 100 includes a restrictor 101 and a body 110 including an inlet 116, a first chamber 102, a second chamber 106, a diaphragm 104, adjustable nozzle 103, o-ring seals 107a-c, reference port 109 and an outlet 105. In some embodiments, during sampling or testing, the outlet 105 of the flow controller 100 can be coupled to a canister that is initially (e.g., substantially) evacuated, thereby providing a negative pressure at the outlet 105 of the flow controller. During testing, in some embodiments, the restrictor 101 and reference port 109 can be fluidly coupled to a pressurized challenge standard or pressurized humid zero air. In some embodiments, during sampling, the restrictor 101 and reference port 109 can be fluidly coupled to the environment of the flow controller 100 and sample canister. Coupling the restrictor 101 and the reference port 109 to the same pressure as each other (e.g., both are coupled to one of the atmosphere, the pressurized challenge standard, or pressurized humid zero air) enables the flow controller 100 to produce the same consistent flow rates during testing and sampling, causing the test setup to accurately reflect performance in the field.

In some embodiments, a gas (e.g., a sample, challenge standard, humidified zero air) can enter the flow controller 100 via flow path 112. The gas can flow through restrictor 101, which can include a filter and glass tube or sapphire orifice, to enter the flow controller 100 body 110 through inlet 116. Inlet 116 can be fluidly coupled to the first chamber 102 of the flow controller 100 body 110, thereby enabling the gas to enter the first chamber 102 through restrictor 101.

In some embodiments, the body 110 of the flow controller 100 can further include nozzle 103. Nozzle 103 can be (e.g., adjustably) positioned relative to diaphragm 104 to allow the vacuum within the first chamber 102 to be just under the pressure at the inlet 116 of the flow controller 100. As the vacuum increases in the first chamber 102 due to the influence of the initially (e.g., substantially) evacuated sample canister coupled to outlet 105, the pressure difference between the first chamber 102 and the second chamber 106 can increase. When the pressure difference between the first chamber 102 and second chamber 106 is large enough, diaphragm 104 can deflect towards the nozzle 103, which can stop the flow of gas into the nozzle 103. In some embodiments, o-ring seals 107a and 107b can seal the flow controller continuously. When the diaphragm 104 deflects to reach the nozzle 103, seal 107c can seal the flow path 114 out of the flow controller 100 through outlet 105, which can seal the first chamber 102 from the influence of the negative pressure at the outlet 105 of the flow controller 100. As gas continues to flow into the first chamber 102 through restrictor 101 and inlet 116, the pressure in the first chamber 102 can increase enough for a leak to occur at o-ring 107c, allowing gas to continue to exit the flow controller 100 through flow path 114. The positive or atmospheric pressure at inlet 116 and negative pressure at outlet 105 can create a sub-atmospheric pressure equilibrium in the first chamber 102, causing a (e.g., substantially) constant flow rate of gas through flow controller 100 and into the sample canister. In some embodiments, once the pressure in the sample canister rises to about the pressure of the first chamber 102 (e.g., due to the gas that was transferred to the sample canister), the flow can decrease. For example, the closer the pressure in the first chamber 102 is to that of atmospheric pressure, the closer to atmospheric pressure the canister pressure can reach while sampling at a constant rate before the flow rate decreases. This is important because it allows more sample to be collected for subsequent analysis, or to allow multiple analyses from the sample canister.

In some embodiments, adjusting the position of the nozzle 103 can adjust the flow rate through the flow controller 100. For example, bringing the nozzle 103 closer to the diaphragm 104 can reduce the pressure differential between the first chamber 102 and the second chamber 106 needed to move diaphragm 104 to a sealing position against o-ring 107c, reducing the pressure differential across restrictor 101, thereby producing a lower flow rate, and causing the time it takes to fill the sample canister to increase. As another example, bringing the nozzle 103 further from the diaphragm 104 can increase the pressure differential between the first chamber 102 and the second chamber 106 needed to move diaphragm 104 to a sealing position against o-ring 107c, increasing the pressure differential across restrictor 101, thereby producing a higher flow rate, and reducing the time it takes to fill the sample canister. In some embodiments, sampling durations in the range of 1 hour to 1 month can be selected by adjusting the nozzle 103 position, changing the restrictor 101 to be more or less restrictive, and/or by changing the size of the canister to be filled.

FIG. 1 shows a how inlet 116 can be coupled to connection 108 when using a pressurized gas (e.g., challenge standard) to perform quality assurance testing to verify the recovery of one or one hundred or more target chemicals in the pressurized gas (e.g., challenge standard), for example. In some embodiments, a pressurized gas (e.g., zero humidified air) can be connected to the flow controller 100 using connection 108 when testing the flow controller 100 for cleanliness. During sampling in the field, connection 108 may not be connected to pressurized gas. Instead, the restrictor 101 and the reference port 109 can both be fluidly coupled to the air in the environment of the flow controller 100, thereby allowing air to enter the flow controller 100 through restrictor 101 and inlet 116 and providing an atmospheric pressure to second chamber 106.

If a pressurized calibration standard is coupled to inlet 116 while reference port 109 is coupled to atmospheric pressure, the flow rate of the calibration standard through the flow controller 100 will be substantially higher than the flow rate of a gas at atmospheric pressure. Elevated flow rates can occur even when the calibration standard at the inlet 116 of the flow controller 100 is at a relatively low pressure, such as about 1 psig above atmospheric pressure. In some embodiments, during sampling of air at atmospheric pressure, the typical pressure in the first chamber 102 may be about 0.3-0.5 psi below the pressure in the second chamber 106. Thus, elevating the pressure of the gas (e.g., the challenge standard) coupled to the inlet 116 of the flow controller 100 by, for example, 1 psig, can increase the flow rate by about 330% compared to the flow rate of a gas at atmospheric pressure at the inlet 116 of the flow controller. Increasing the flow rate in this way can decrease the residence time in the flow controller 100 (e.g., by a factor of about 3.3), which can reduce the extent to which compounds in the flow controller 100 interact or react with interior surfaces of the flow controller 100. For example, a flow controller 100 calibrated to produce a flow rate of 3.3 cc/min when the gas at the inlet 116 is at atmospheric pressure can produce a flow rate of about 10 cc/min when the gas at the inlet 116 is about 1 psig above atmospheric pressure, which can be considered a substantially different flow rate. Conducting a test of the flow controller 100 with such a substantial flow rate difference between testing and sampling may not be able to properly validate the performance of the flow controller 100. For example, due to the increased flow rate, it may appear as though the flow controller 100 is less reactive and/or absorptive than it would be during sampling.

In some embodiments, the disclosed flow controller 100 is capable of operating with (e.g., substantially) the same flow rate during testing and sampling. For example, flow controller 100 includes reference port 109, which can be coupled to the pressurized supply of challenge standard or humidified zero air. Coupling the flow controller 100 to the pressurized supply of challenge standard or humidified zero air at the reference port 109 and the inlet 116 can balance the pressures in the first chamber 102 and the second chamber 106, which causes the flow controller 100 to produce (e.g., substantially) the same flow rates that would occur during sampling. It should be appreciated that, during sampling, both the inlet 116 of the flow controller 100 and the reference port 109 of the flow controller 100 are fluidly coupled to gas (e.g., ambient air) at atmospheric pressure, which causes the pressures in the first chamber 102 and the second chamber 106 to be balanced. In some embodiments, pressure balancing during testing and calibration can cause the flow rate during testing and calibration to be within about 10% of the flow rate during sampling. This performance is an improvement compared to the 330% increase in flow rate that would occur if the reference port 109 were not coupled to the pressurized challenge standard or pressurized humidified zero air during testing and calibration. Balancing the flow rates during sampling and calibration/testing allows the residence time in the flow controller 100 to be substantially the same during sampling and calibration/testing when using a 1-2 psig standard at the flow controller 100 inlet 116 (e.g., via restrictor 101) and reference port 109.

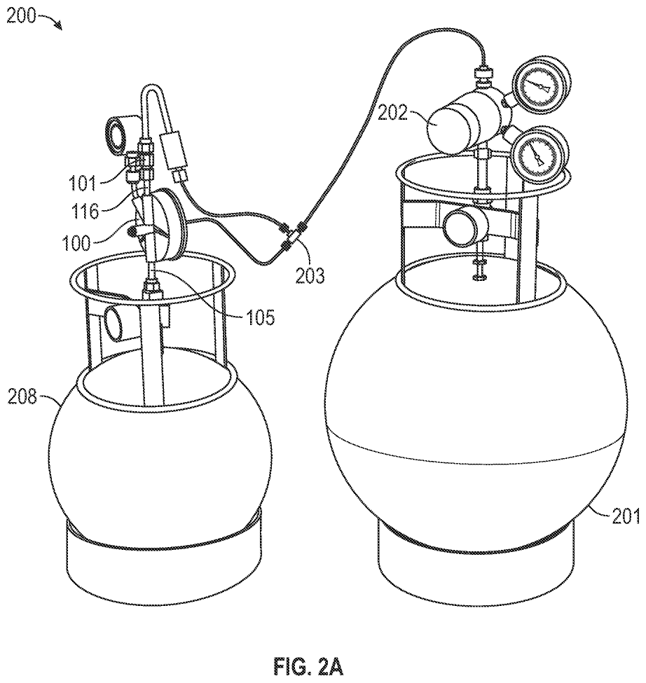

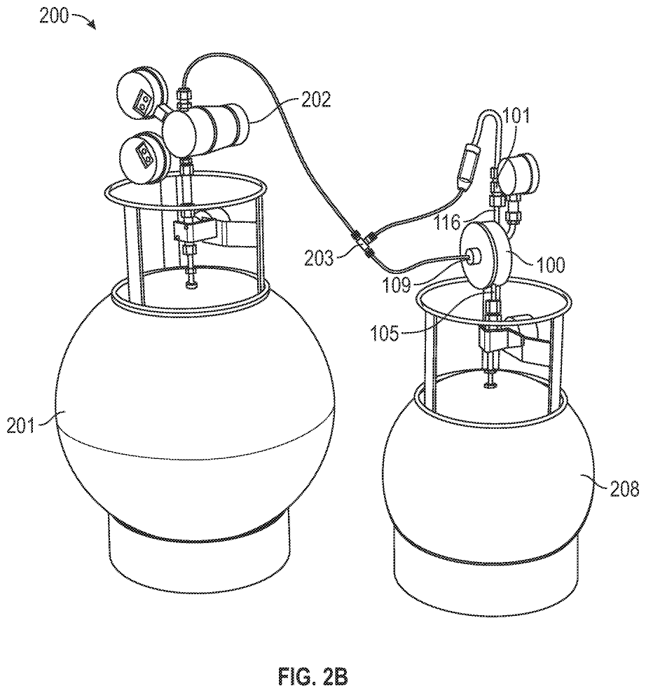

FIGS. 2A-2B illustrate a test setup 200 including a flow controller 100 according to some embodiments of the disclosure. Test setup 200 can include a canister 201 containing a challenge standard or humidified zero air, a pressure regulator 202, tee 203, sample canister 208, and flow controller 100. In some embodiments, flow controller 100 can include (e.g., one or more, all of) the components described above with reference to FIG. 1.

In some embodiments, canister 201 can contain a calibration standard including 1 to 200 compounds at known concentrations (e.g., typically at PPB to sub-PPB levels) and water, such that the calibration standard has about 40-50% Relative Humidity. In some embodiments, canister 201 can contain humidified zero air. In some embodiments, the gas within canister 201 has a pressure in the range of 5 to 100 psig. Pressure regulator 202 can be coupled to the outlet of canister 201 to reduce the pressure of the gas to 1-2 psig. In some embodiments, tee 203 can be used to fluidly couple the gas in canister 201 to both the inlet 116 of the flow controller 100 (e.g., via restrictor 101), and the reference port 109 of the flow controller 100, thereby balancing the pressures on both sides of the diaphragm 104 of the flow controller 100. The outlet 105 of the flow controller 100 can be coupled to sample canister 208. In some embodiments, at the beginning of the testing or calibration procedure, the sample canister can be (e.g., fully, substantially) evacuated (e.g., the sample canister 208 can have a negative pressure). The negative pressure of the sample canister 208 can pull gas (e.g., challenge standard or humidified zero air from canister 201) through the flow controller 100 into the sample canister 208. Gas from canister 201 can enter the flow controller 100 at inlet 101 and traverse the flow controller 100 to enter sample canister 208 at a known, stable flow rate. In some embodiments, the lines coupling the gas to the flow controller 100 are typically chromatographic grade, and can be ceramic-lined stainless steel to reduce any potential for surface interactions. The various connections (e.g., between transfer lines, canisters 201 and 208, the flow controller 100, tee 203, etc.) can be made in any number of ways, including using o-rings, compression fittings, or other leak tight connections.

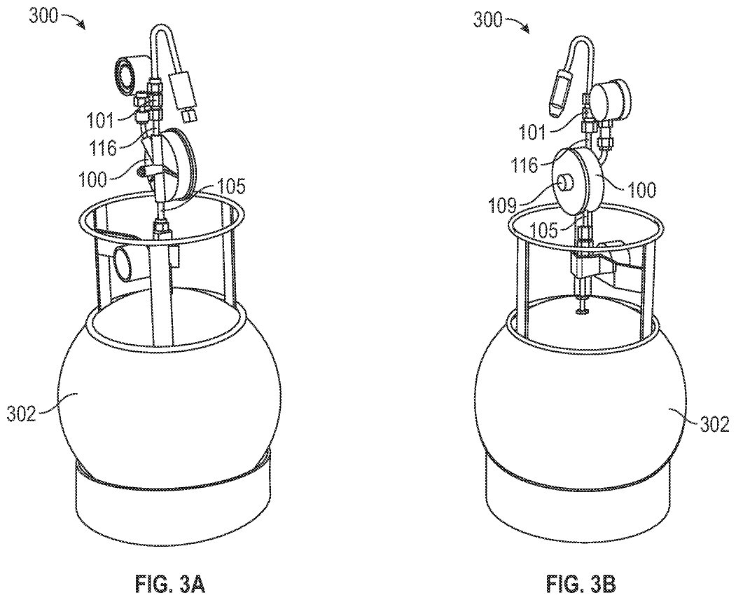

FIGS. 3A-3B illustrate a sampling system 300 including flow controller 100 according to some embodiments of the disclosure. The sampling system 300 can include flow controller 100 described above with reference to FIG. 1 and sampling canister 302. In some embodiments, during sampling, the inlet 116 and the reference port 109 of the flow controller 100 can be open to the environment of the sampling system 300. Thus, both sides of the diaphragm 104 of the flow controller 100 can be at atmospheric pressure and the air in the environment of the sampling system 300 can enter the flow controller 100. The outlet 105 of the flow controller can be coupled to the sample canister 302. In some embodiments, sample canister 302 can be the same as or similar to the sample canister 208 described above with reference to FIGS. 2A-2B. In some embodiments, at the start of the sampling process, the sample canister 302 can be (e.g., fully, substantially) evacuated and can therefore provide a negative pressure at the outlet 105 of the flow controller 100. The negative pressure at the outlet 105 of the flow controller 100 can pull gas (e.g., ambient air) through the flow controller 100 and into the sample canister 302.

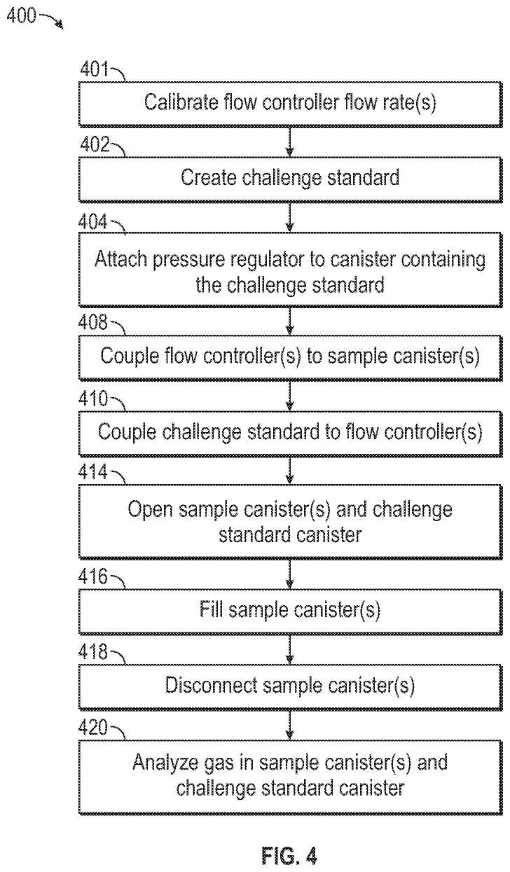

FIG. 4 illustrates a method 400 of challenging a flow controller for proper recovery of target compounds of interest according to some embodiments of the disclosure. In some embodiments, method 400 can be performed using flow controller 100 described above with reference to FIG. 1 and system 200 described above with reference to FIGS. 2A-2B. In some embodiments, the steps of method 400 can be performed in the order indicated in FIG. 4. In some embodiments, the order of one or more steps of method 400 can be altered. Moreover, in some embodiments, one or more steps of method 400 can be skipped or repeated.

In some embodiments, method 400 includes calibrating 401 one or more flow controllers 100 to be challenged. The flow rate(s) of the flow controller(s) 100 to be challenged should be adjusted to be (e.g., substantially) equal to the flow rate(s) to be used during sampling. In some embodiments, the flow rate(s) through the flow controller(s) 100 can be adjusted by adjusting the position of the nozzle 103 of the flow controller(s) 100, the duration of the test, the volume of the sample canister(s) 208 used during the test, and the restrictor(s) 101 used during the test. In some embodiments, an automated flow controller calibration system can calibrate the flow rate(s) of the flow controller(s) 100 to be tested.

In some embodiments, the method 400 includes creating 402 a challenge standard. In some embodiments, the challenge standard includes a mixture of all target compounds (e.g., to be identified in a subsequent analysis of ambient air) at 0.2-0.5 PPBv. The challenge standard can be created in a 15 L ceramic-coated (e.g., Silonite) canister 201 at 20-50 psig using static or dynamic dilution. VOC-free water can be added to the canister 201 to bring the relative humidity of the challenge mixture to 40-50%.

In some embodiments, method 400 includes attaching 404 a pressure regulator 202 to the canister 201 containing the challenge standard. In some embodiments, the pressure regulator 202 is a dual stage, Ultra High Purity stainless steel regulator configured to produce a pressure in the range of 1-2 psig at its outlet.

In some embodiments, method 400 includes coupling 408 the flow controller(s) 100 to sample canister(s) 208. In some embodiments, the volume of the sample canister 208 used during testing can differ from the volume of the canister to be used for sampling. In some embodiments, the flow rate during testing is the same as the flow rate during sampling. For example, rather than using a 6 L canister to collect a 2 week sample at 0.35 cc/min, a 0.6 L canister can be used to fill the canister at the same flow rate in under 36 hours. In some embodiments, coupling the flow controller(s) 100 to sample canister(s) 208 can create a vacuum in the transfer lines coupling the challenge standard to the flow controller(s) 100. The vacuum can be pulled by opening and subsequently closing the isolation valve on the receiving canister 208 (or just one of the many receiving canisters) to pull a vacuum on the transfer lines. If no leaks are present in the system (e.g., transfer lines, flow controller, pressure regulator(s), canisters, fittings, etc.), the vacuum should be maintained.

In some embodiments, method 400 includes coupling 410 the challenge standard to the flow controller(s) 100. In some embodiments, ceramic (Silonite) coated stainless steel, ⅛″ OD tubing can be used to make the connections. For example, the tubing can be coupled to the output of the pressure regulator 202 and a (e.g., ceramic coated stainless steel) tee 203, which can be coupled to the inlet 116 and reference port 109 of the pressure regulator 100. In some embodiments, tee 203 can be replaced with a cross, which can facilitate coupling of more than one flow controller 100 to the challenge standard. For example, one position of the cross can be coupled to the challenge standard, a second position of the cross can be coupled to the inlet 116 of a first flow controller 100, a third position of the cross can be coupled to the reference port 109 of the first flow controller 100, and a fourth position of the cross can be coupled to another cross, which in turn can be coupled to the inlet and reference port of a second flow controller. In some embodiments, a plurality (e.g., up to 5) flow controllers can be connected to the same challenge standard to be simultaneously challenged.

In some embodiments, method 400 includes opening 414 the sample canister(s) 208 coupled to the flow controller(s) 100 to be challenged and the canister 201 containing the challenge standard. In some embodiments, the canister(s) 208 coupled to the flow controller(s) 100 to be challenged are opened before opening the canister 201 containing the challenge standard. In some embodiments, the outlet pressure of the pressure regulator 202 coupled to the canister 201 containing the challenge standard is set to 1-2 psi (e.g., before or after opening canister 201).

In some embodiments, method 400 includes filling 416 the sample canister(s) 208 coupled to the flow controller(s) 100 to be challenged. In some embodiments, the negative pressure of the sample canister(s) 208 can drive the flow of the challenge standard into the canister(s) 208. The flow can be allowed to continue until the sample canister(s) 208 are full.

In some embodiments, method 400 includes disconnecting 418 the sample canister(s) 208 from the rest of the testing system 200. In some embodiments, the sample canister(s) 208 are closed prior to disconnecting them to avoid contamination of the gas contained in the sample canister(s) 208.

In some embodiments, method 400 includes analyzing the gas in the sample canister(s) 208 and the gas (e.g., challenge standard) remaining in canister 201. In some embodiments, the gas contained in canisters 208 and 201 can be preconcentrated using a preconcentration system, followed by analysis (e.g., by GCMS). In some embodiments, the concentrations of the target compounds in the receiving canister(s) 208 should be within 15-20% of the concentrations of the target compounds in the challenge canister 201, provided that the compounds of interest did not break down or remain in the flow controller(s) 100.

In some embodiments, the flow controller(s) can be tested for cleanliness using method 400. In some embodiments, first, the flow controller(s) can be cleaned by flushing them with humid nitrogen and/or subjecting the flow controller(s) to a vacuum to extract residual contaiminants. Next, method 400 can be performed using a humid zero air blank instead of a challenge standard. After filling, the contents of the receiving canister(s) 208 can be analyzed to confirm that the concentrations of the compounds of interest have not increased by more than 0.02 PPBv (e.g., according to EPA method TO-15A).

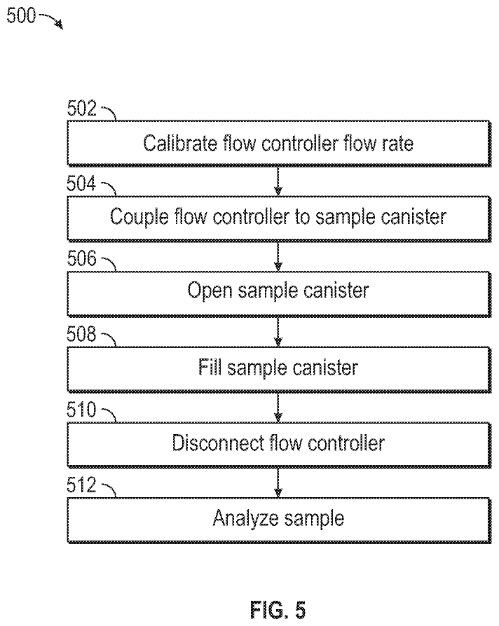

FIG. 5 illustrates a method 500 of collecting a sample using flow controller 100 according to some embodiments of the disclosure. In some embodiments, method 500 can be performed using flow controller 100 described above with reference to FIG. 1 and system 300 described above with reference to FIGS. 3A-3B. In some embodiments, the steps of method 500 can be performed in the order indicated in FIG. 5. In some embodiments, the order of one or more steps of method 500 can be altered. Moreover, in some embodiments, one or more steps of method 500 can be skipped or repeated.

In some embodiments, method 500 includes calibrating 502 the flow rate of the flow controller 100 to be used to collect the sample. The flow rate of the flow controller 100 to be used for sampling can be adjusted to be (e.g., substantially) equal to the flow rate used during testing/challenging. In some embodiments, the flow rate through the flow controller 100 can be altered by adjusting the position of the nozzle 103 of the flow controller, the duration of the sample collection process, the volume of the sample canister 302 used during sample collection, and the restrictor(s) 101 used during sample collection. In some embodiments, an automated flow controller calibration system can calibrate the flow rate of the flow controller 100.

In some embodiments, method 500 includes coupling 504 the flow controller 100 to the sample canister 302. In some embodiments, the outlet 105 of the flow controller 100 is connected to the sample canister 302. During sampling, the inlet 116 and the reference port 109 of the flow controller 100 can be open to the environment of the sampling system 300.

In some embodiments, method 500 includes opening 506 the sample canister 302. In some embodiments, the sample canister 302 is (e.g., substantially) evacuated prior to the sampling process. Thus, opening the sample canister 302 can provide a negative pressure at the outlet 105 of the flow controller 100, driving the flow of the air in the environment of the sampling system 300 through the flow controller 100 and into the canister 302.

In some embodiments, method 500 includes filling 508 the sample canister 302. In some embodiments, the negative pressure of the canister 302 caused by the initial (e.g., substantial) evacuation of the canister 302 can drive the flow of ambient air through the flow controller 100 into the canister 302. Sampling can occur until the canister 302 has been filled, for example. In some embodiments, once there is enough gas (e.g., ambient air) in canister 302 to balance the pressure in canister 302 with the pressure in the flow controller 100, the pressure of the canister 302 will no longer drive a flow of ambient air through the flow controller 100 and into the canister 302.

In some embodiments, method 500 includes disconnecting 510 the flow controller 100 from the sample canister 302. In some embodiments, the canister 302 is closed prior to disconnecting the flow controller 100 to prevent additional air from entering canister 302.

In some embodiments, method 500 includes analyzing 512 the sample in the sample canister 302. In some embodiments, the canister 302 can be coupled to a sample preconcentration system to preconcentrate the sample prior to analysis (e.g., by GCMS).

Therefore, according to the above, some embodiments of the disclosure relate to a method comprising: during a calibration process: coupling an inlet of a flow controller and a reference port of the flow controller to a gas having a respective pressure above atmospheric pressure; coupling an outlet of the flow controller to a first canister, the first canister initially having a first negative pressure; and filling the first canister at a first flow rate; and during a sampling process: coupling the inlet of the flow controller and the reference port of the flow controller to air in an environment of the flow controller; coupling the outlet of the flow controller to a second canister, the second canister initially having a second negative pressure; and filling the second canister at a second flow rate that is within 10% of the first flow rate. Additionally or alternatively, in some embodiments the respective pressure is 1-2 psig above atmospheric pressure. Additionally or alternatively, in some embodiments a pressure of the air in the environment of the flow controller is atmospheric pressure. Additionally or alternatively, in some embodiments the method further includes after filling the first canister, analyzing contents of the first canister to test the flow controller; and after filling the second canister, analyzing contents of the second canister to perform analysis of the air in the environment of the flow controller. Additionally or alternatively, in some embodiments the flow controller includes a first chamber fluidly coupled to the inlet of the flow controller and the outlet of the flow controller; a second chamber fluidly coupled to reference port of the flow controller; and a diaphragm disposed between the first chamber and the second chamber. Additionally or alternatively, in some embodiments the method further includes adjusting the first flow rate by adjusting a position of an adjustable nozzle of the flow controller; and adjusting the second flow rate by adjusting a position of an adjustable nozzle of the flow controller. Additionally or alternatively, in some embodiments while filling the first canister, the first flow rate is substantially constant, and while filling the second canister, the second flow rate is substantially constant.

Some embodiments of the disclosure relate to a flow controller comprising an inlet; an outlet configured to be coupled to a canister initially having a negative pressure; a reference port; a first chamber fluidly coupled to the inlet of the flow controller and the outlet of the flow controller; a second chamber fluidly coupled to the reference port of the flow controller; and a diaphragm disposed between the first chamber and the second chamber, wherein: the diaphragm is configured to deflect in response to a pressure difference between the first chamber and the second chamber, the inlet and the reference port are configured to be coupled to a gas having a respective pressure in the range of atmospheric pressure to 2 psig above atmospheric pressure, and the flow controller is configured to produce a flow rate within 10% of a respective flow rate irrespective of the respective pressure of the gas, the respective pressure in the range of atmospheric pressure to 2 psig above atmospheric pressure. Additionally or alternatively, in some embodiments the flow controller includes an adjustable nozzle that, when adjusted, adjusts the flow rate of the flow controller. Additionally or alternatively, in some embodiments one or more interior surfaces of the flow controller are substantially inert. Additionally or alternatively, in some embodiments the flow controller further includes a restrictor coupled to the inlet of the flow controller. Additionally or alternatively, in some embodiments the flow rate is produced while filling the canister, and the flow rate remains within +/−10% of being constant while filling the canister.

Some embodiments of the disclosure relate to a system comprising: a first canister initially having a negative pressure; a second canister containing a first gas having a respective positive pressure not exceeding 2 psig; a flow controller comprising: an inlet; an outlet; a reference port; a first chamber fluidly coupled to the inlet of the flow controller and the outlet of the flow controller; a second chamber fluidly coupled to the reference port of the flow controller; and a diaphragm disposed between the first chamber and the second chamber, wherein: the diaphragm is configured to deflect in response to a pressure difference between the first chamber and the second chamber, the outlet of the flow controller is coupled to the first canister, the inlet of the flow controller and the reference port of the flow controller are coupled to the second canister, and while the inlet of the flow controller and the reference port of the flow controller are coupled to the second canister, the flow controller is configured to produce a flow rate within 10% of a respective flow rate produced by the flow controller while the inlet of the flow controller and the reference port of the flow controller are coupled to a second gas at atmospheric pressure. Additionally or alternatively, in some embodiments the first canister is configured to collect a gas to be analyzed to test the flow controller. Additionally or alternatively, in some embodiments the flow controller further includes an adjustable nozzle that, when adjusted, adjusts the flow rate of the flow controller. Additionally or alternatively, in some embodiments one or more interior surfaces of the flow controller are substantially inert. Additionally or alternatively, in some embodiments the system further includes a restrictor coupled to the inlet of the flow controller. Additionally or alternatively, in some embodiments the flow rate is produced while filling the first canister, and the flow rate remains within +/−10% of being constant while filling the first canister.

Although examples have been fully described with reference to the accompanying drawings, it is to be noted that various changes and modifications will become apparent to those skilled in the art. Such changes and modifications are to be understood as being included within the scope of examples of this disclosure as defined by the appended claims.

1. A method comprising:
during a calibration process:
coupling an inlet of a flow controller and a reference port of the flow controller to a gas having a respective pressure above atmospheric pressure;
coupling an outlet of the flow controller to a first canister, the first canister initially having a first negative pressure; and
filling the first canister at a first flow rate; and
during a sampling process:
coupling the inlet of the flow controller and the reference port of the flow controller to air in an environment of the flow controller;
coupling the outlet of the flow controller to a second canister, the second canister initially having a second negative pressure; and
filling the second canister at a second flow rate that is within 10% of the first flow rate.

2. The method of claim 1, wherein the respective pressure is 1-2 psig above atmospheric pressure.

3. The method of claim 1, wherein a pressure of the air in the environment of the flow controller is atmospheric pressure.

4. The method of claim 1, further comprising:
after filling the first canister, analyzing contents of the first canister to test the flow controller; and
after filling the second canister, analyzing contents of the second canister to perform analysis of the air in the environment of the flow controller.

5. The method of claim 1, wherein the flow controller includes:
a first chamber fluidly coupled to the inlet of the flow controller and the outlet of the flow controller;
a second chamber fluidly coupled to reference port of the flow controller; and
a diaphragm disposed between the first chamber and the second chamber.

6. The method of claim 1, further comprising:
adjusting the first flow rate by adjusting a position of an adjustable nozzle of the flow controller; and
adjusting the second flow rate by adjusting a position of an adjustable nozzle of the flow controller.

7. The method of claim 1, wherein:
while filling the first canister, the first flow rate is substantially constant, and
while filling the second canister, the second flow rate is substantially constant.

8. A flow controller, comprising:
an inlet;
an outlet configured to be coupled to a canister initially having a negative pressure;
a reference port;
a first chamber fluidly coupled to the inlet of the flow controller and the outlet of the flow controller;
a second chamber fluidly coupled to the reference port of the flow controller; and
a diaphragm disposed between the first chamber and the second chamber, wherein:
the diaphragm is configured to deflect in response to a pressure difference between the first chamber and the second chamber,
the inlet and the reference port are configured to be coupled to a gas having a respective pressure in the range of atmospheric pressure to 2 psig above atmospheric pressure, and
the flow controller is configured to produce a flow rate within 10% of a respective flow rate irrespective of the respective pressure of the gas, the respective pressure in the range of atmospheric pressure to 2 psig above atmospheric pressure.

9. The flow controller of claim 8, further comprising an adjustable nozzle that, when adjusted, adjusts the flow rate of the flow controller.

10. The flow controller of claim 8, wherein one or more interior surfaces of the flow controller are substantially inert.

11. The flow controller of claim 8, further comprising a restrictor coupled to the inlet of the flow controller.

12. The flow controller of claim 8, wherein the flow rate is produced while filling the canister, and the flow rate remains within +/−10% of being constant while filling the canister.

13. A system comprising:
a first canister initially having a negative pressure;
a second canister containing a first gas having a respective positive pressure not exceeding 2 psig;
a flow controller comprising:
an inlet;
an outlet;
a reference port;
a first chamber fluidly coupled to the inlet of the flow controller and the outlet of the flow controller;
a second chamber fluidly coupled to the reference port of the flow controller; and
a diaphragm disposed between the first chamber and the second chamber,
wherein:
the diaphragm is configured to deflect in response to a pressure difference between the first chamber and the second chamber,
the outlet of the flow controller is coupled to the first canister,
the inlet of the flow controller and the reference port of the flow controller are coupled to the second canister, and
while the inlet of the flow controller and the reference port of the flow controller are coupled to the second canister, the flow controller is configured to produce a flow rate within 10% of a respective flow rate produced by the flow controller while the inlet of the flow controller and the reference port of the flow controller are coupled to a second gas at atmospheric pressure.

14. The system of claim 13, wherein the first canister is configured to collect a gas to be analyzed to test the flow controller.

15. The system of claim 13, wherein the flow controller further includes an adjustable nozzle that, when adjusted, adjusts the flow rate of the flow controller.

16. The system of claim 13, wherein one or more interior surfaces of the flow controller are substantially inert.

17. The system of claim 13, further comprising a restrictor coupled to the inlet of the flow controller.

18. The system of claim 13, wherein the flow rate is produced while filling the first canister, and the flow rate remains within +/−10% of being constant while filling the first canister.
